(19) United States
(12) Patent Application Publication
(10) Pub. No.: US 2018/0147141 A1
(43) Pub. Date: May 31, 2018

(54) CHEWING GUM COMPOSITION COMPRISING CANNABINOIDS AND GABAPENTIN

(71) Applicant: AXIM Biotechnologies, Inc., New York, NY (US)

(72) Inventors: Lekhram CHANGOER, Ridderkerk (NL); George ANASTASSOV, New York, NY (US)

(21) Appl. No.: 15/824,184

(22) Filed: Nov. 28, 2017

Related U.S. Application Data
(60) Provisional application No. 62/427,797, filed on Nov. 29, 2016.

(51) Int. Cl. A61K 9/68; A61K 31/352; A61K 31/195; A61K 47/26; A61K 47/38; A61K 47/36; A61K 47/12; A61K 47/02; A61K 47/10; A61K 47/22

(52) U.S. Cl. CPC A61K 9/0058; A61K 31/352; A61K 31/195; A61K 47/26; A61K 47/22; A61K 47/36; A61K 47/12; A61K 47/02; A61K 47/10; A61K 47/38

(57) ABSTRACT

A chewing gum composition comprising cannabinoids or derivatives thereof and gabapentin is provided. The chewing gum composition is formulated to provide controlled release of cannabinoids and gabapentin during mastication. Methods to provide post-herpetic neuralgia and restless leg syndrome treatment using the chewing gum composition according to this invention are also provided.

### CROSS-REFERENCE TO RELATED APPLICATIONS

This claims the benefit of U.S. Provisional Application No. 62/427,797, filed Nov. 29, 2016. Each of the above-referenced patent applications is incorporated by reference in its entirety.

### BACKGROUND OF THE INVENTION

### Field of the Invention

Post-herpetic neuralgia, restless leg syndrome, and other neuropathic pain treatments have received increased interest in recent years due to the increased prevalence of these conditions. This invention concerns a chewing gum composition for the treatment of post-herpetic neuralgia and restless leg syndrome, wherein active ingredients may be released in a controlled manner. The chewing gum composition is a delivery system which provides controlled release of active pharmaceutical ingredients and dual action mechanism for treatment of the above conditions.

### Description of the Related Technology

The cannabis plant has many naturally occurring substances that are of great interest in the fields of science and medicine. Isolated compounds from the cannabis plant include Δ⁹-tetrahydrocannabinol (THC), cannabidiol (CBD), cannabichromene (CBC), cannabigerol (CBG), cannabidivarin (CBDV), among other compounds. While THC has psychoactive effects, CBD, CBC, CBG, and CBDV do not. Isolated compounds from the cannabis plant are called cannabinoids. There are a total of one hundred and forty one (141) cannabinoids that have been isolated from the cannabis plant. Many researchers have confirmed the medicinal value of cannabinoids. Cannabinoids have been investigated for possible treatment of seizures, nausea, vomiting, lack of appetite, pain, arthritis, inflammation, and other conditions.

The IUPAC nomenclature of THC is (−)-(6aR,10aR)-6,6,9-trimethyl-3-pentyl-6a,7,8,10a-tetrahydro-6H-benzo[c]chromen-1-ol. CBD's IUPAC nomenclature is 2-((1S,6S)-3-methyl-6-(prop-1-en-2-yl)cyclo-hex-2-enyl)-5-pentylbenzene-1,3 -diol). CBC has the IUPAC nomenclature of 2-methyl-2-(4-methylpent-3-enyl)-7pentyl-5-chromenol. These are among the most prominent compounds in the family of compounds extracted from the cannabis plant referred to as cannabinoids.

Cannabinoids may be isolated by extraction or cold pressing of cannabis plants. Plants in the cannabis genus include Cannabis sativa, Cannabis ruderalis, and Cannabis indica. These plants are the natural sources of cannabinoids. Cannabinoids are also available in synthetic forms. Methods to synthesize cannabinoids in lab settings were discovered and are still currently practiced. Synthetic cannabinoids are more targeted, in that the synthetic compound usually comes isolated without other cannabinoids mixed in.

Cannabidiol is a major phytocannabinoid, accounting for up to 40% of the plant's extract. CBD is a CB-1 receptor antagonist, while THC is a CB-1 receptor agonist. A 2010 research found that cannabis strains with higher concentration of CBD did not produce the short-term memory impairment normally seen in high THC cannabis strains, a characteristic attributed to the CB-1 receptor antagonist nature of CBD. CBD is considered to have a wider scope of medical applications than THC.

Gabapentin has an IUPAC name of 1-(aminomethyl)cyclohexaneacetic acid. Gabapentin was initially synthesized to mimic the chemical structure of the neurotransmitter gamma-aminobutyric acid (GABA), and it in fact has a similar chemical structure to GABA. However, gabapentin has not been shown to bind to GABA receptors at concentrations at or below 1 millimolar. Gabapentin modulates the action of glutamate decarboxylase (GAD) and branched chain aminotransferase (BCAT), two enzymes involved in GABA biosynthesis, which may have an effect on GABA biosynthesis and/or GABA concentration.

Gabapentin activity may involve interaction with voltage-gated calcium channels. By binding to the α2δ subunit (both 1 and 2), a protein in the voltage-dependent calcium channel complex, gabapentin reduces calcium currents after chronic but not acute application, via an effect on trafficking of voltage-dependent calcium channels in the central nervous system.

Gabapentin has been marketed under the brand name Neurontin. Since becoming a generic drug in 2004, gabapentin has been marketed under other brand names. Gabapentin is commonly packaged in an oral pill or an oral liquid solution.

### ABBREVIATIONS

BCAT: branched chain aminotransferase
CBC: cannabichromene
CBD: cannabidiol
CBDV: cannabidivarin
CBG: cannabigerol
GABA: gamma-aminobutyric acid
GAD: glutamate decarboxylase
IUPAC: International Union of Pure and Applied Chemistry
THC: Δ⁹-tetrahydrocannabinol

### SUMMARY

The present invention relates to a chewing gum composition comprising cannabinoids or derivatives thereof and gabapentin. Cannabinoids or derivatives thereof and gabapentin are under controlled release during mastication. This invention further relates to the use of this chewing gum composition in treating post-herpetic neuralgia, restless leg syndrome, and other neuropathic pain conditions.

This invention provides a chewing gum composition comprising, based on total weight of the composition:

0.125 to 7.5% by weight of at least one cannabinoid;

5 to 15% by weight of gabapentin;

20 to 80% by weight of a gum base;

5 to 35% by weight of at least one buffering agent selected from the group consisting of acetates, glycinates, phosphates, carbonates, glycerophosphates, citrates, and borates;

1 to 10% by weight of at least one flavoring agent;

1 to 10% by weight of at least one flavoring agent selected from the group consisting of peppermint, cinnamon, watermelon, and spearmint;

1 to 65% by weight of at least one sweetening agent selected from the group consisting of isomalt, sorbitol, stevia, maltitol, and xylitol; and

at least one anti-oxidant selected from the group consisting of ascorbyl palminate and sodium ascorbate.

This invention further provides a chewing gum composition according to embodiments, wherein the at least one cannabinoid is cannabidiol, cannabichromene, cannabigerol, cannabidivarin, derivatives thereof, or their acid metabolites.

This invention further provides a chewing gum composition according to embodiments, wherein the at least one cannabinoid is Δ⁹-tetrahydrocannabinol.

This invention further provides a chewing gum composition according to embodiments, wherein Δ⁹-tetrahydrocannabinol is present at 0.125 to 1% by weight of the total composition.

This invention further provides a chewing gum composition according to embodiments, wherein the at least one cannabinoid is provided in combination with at least one suitable carrier selected from the group consisting of sugar alcohol, microcrystalline cellulose derivatives, dextran, agarose, agar, pectin, alginate, xanthan, chitosan, and starch.

This invention further provides a chewing gum composition according to embodiments, wherein the at least one cannabinoid is provided in freeze dried form.

This invention further provides a chewing gum composition according to embodiments, wherein the at least one cannabinoid is provided in internal voids within a suitable solid carrier.

This invention further provides a chewing gum composition according to embodiments, wherein the at least one cannabinoid is provided in combination with a carrier in a granule within the gum matrix.

This invention further provides a chewing gum composition according to embodiments, wherein the at least one cannabinoid is procured from natural sources or synthetic.

This invention further provides a chewing gum composition according to embodiments, wherein the at least one flavoring agent is selected from the group consisting of peppermint, spearmint, cinnamon, licorice, cherry, orange, peach, and watermelon.

This invention further provides a chewing gum composition according to embodiments, wherein the at least one cannabinoid and the gabapentin are provided as microencapsulated or nanoencapsulated particles.

This invention further provides a chewing gum composition according to embodiments, wherein the nanoencapsulated or microencapsulated gabapentin or cannabinoid particles are liposomal particles.

This invention further provides a chewing gum composition according to embodiments, further comprising at least one preservative and at least one antioxidant.

This invention further provides a chewing gum composition according to embodiments, wherein the preservative is citric acid.

This invention further provides a chewing gum composition according to embodiments, further comprising at least one pharmaceutically acceptable excipient selected from the group consisting of fillers, disintegrants, binders, and lubricants.

This invention further provides a chewing gum composition according to embodiments, further comprising silicon dioxide or magnesium stearate.

This invention provides a method to treat post-herpetic neuralgia in a mammal in need thereof, comprising administering to the mammal a chewing gum composition according to embodiments, wherein the mammal receives the chewing gum administration 1 to 6 times a day.

This invention provides a method to treat restless leg syndrome in a mammal in need thereof, comprising administering to the mammal a chewing gum composition according to embodiments, wherein the mammal receives the chewing gum administration 1 to 6 times a day.

### DETAILED DESCRIPTION OF CERTAIN INVENTIVE EMBODIMENTS

This present invention is capable of being embodied in various forms. The description below of several embodiments is made with the understanding that the present disclosure is to be considered as an exemplification of the claimed subject matter, and is not intended to limit the attached claims to the specific embodiments illustrated. The headings used throughout this disclosure are provided for convenience only and are not to be construed to limit the claims in any way. Embodiments illustrated under any heading may be combined with embodiments illustrated under any other heading.

As used herein, the verb “to comprise” in this description, claims, and other conjugations are used in its non-limiting sense to mean those items following the word are included, but items not specifically mentioned are not excluded.

Reference to an element by the indefinite article “a” or “an” does not exclude the possibility that more than one of the elements are present, unless the context clearly requires that there is one and only one of the elements. The indefinite article “a” or “an” thus usually means “at least one.” Additionally, the words “a” and “an” when used in the present document in concert with the words “comprising” or “containing” denote “one or more.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

All numerical ranges given herein are by way of examples to better illustrate the invention embodiments and shall not be construed to limit the invention embodiments to the given numeric values.

Embodiments of this application relate to a chewing gum composition comprising cannabinoids and gabapentin, wherein cannabinoids and gabapentin are incorporated into the chewing gum for controlled release. The chewing gum composition may be consumed by a mammal, such as a human, for treatment of post-herpetic neuralgia, restless leg syndrome, and other neuropathic pain conditions.

In embodiments, the chewing gum composition may comprise at least one cannabinoid. Cannabinoids in these embodiments may be A⁹-tetrahydrocannabinol (THC), cannabichromene (CBC), cannabigerol (CBG), cannabidivarin (CBDV), cannabidiol (CBD), other cannabinoids, derivatives thereof, their acid metabolites, or a combination of cannabinoids and/or their acid metabolites and/or derivatives thereof.

In embodiments, the chewing gum composition may comprise 0.125-7.5% by weight of at least one cannabinoid or derivatives thereof, based on total weight of the composition. In a 2 gram chewing gum piece, cannabinoids or derivatives thereof may comprise 2.5-150 mg. In embodiments wherein the cannabinoid is THC or derivatives thereof, the chewing composition may comprise 0.125-1.0% by weight of THC or derivatives thereof based on total weight of the composition. In a 2 g chewing gum piece, THC may comprise 2.5-20 mg. Where a combination of cannabinoids is used in this chewing gum composition, THC or derivatives thereof may be present at no more than 20 mg in each piece of chewing gum.

Cannabinoids in the chewing gum composition according to embodiments may be synthetic or procured from natural source. Natural sources of cannabinoids may be from cannabis plants, hemp plants, or other organisms capable of producing cannabinoids. Organisms capable of producing cannabinoids may be genetically modified. Where cannabinoids are from natural sources, a combination of cannabinoids may be present at different concentration. The sources may be chosen such that a cannabinoid may be present as the major cannabinoid, such as CBD, CBG, or THC.

Synthetic cannabinoids may be synthesized by methods known in the art. Synthetic cannabinoids are purer, such that only one cannabinoid may be present. A combination of cannabinoids may be provided at ratios as desired. This may be done to achieve the desired concentrations for the various synthetic cannabinoids.

In embodiments, cannabinoids may be provided in a solid material composed of an edible solid, such as a sugar alcohol, to prevent binding with the gum base. Other solids suitable for embedding cannabinoids are contemplated, such that cannabinoids or derivatives thereof are provided within internal voids of solid materials. Alternatively, cannabinoids or derivatives thereof may be provided in a granule embedded into the gum matrix. Cannabinoids or derivatives thereof provided in these manners may improve cannabinoid release during mastication of the chewing gum according to embodiments.

Other suitable carriers which may be combined with cannabinoids before inclusion into the gum matrix may include certain celluloses such as microcrystalline cellulose derivatives, dextran, agarose, agar, pectin, alginate, xanthan, chitosan, or starch. The combination of cannabinoids and suitable carriers may result in cannabinoids being present within internal voids of these carriers.

Providing cannabinoids by combining with a suitable carrier or by providing cannabinoids in a capsule within the gum matrix may enable controlled release of cannabinoids during chewing of the chewing gum composition.

In embodiments, cannabinoids or derivatives thereof may also be provided in microencapsulated or nanoencapsulated form or in freeze dried form. Microencapsulated, nanoencapsulated, or freeze dried cannabinoids may improve the chewing gum's taste, prevent binding with the gum matrix, control cannabinoid release during mastication, and further improve bioavailability of the cannabinoids once entering the gastrointestinal tract.

In the chewing gum composition according to embodiments, cannabinoids may be provided in encapsulated form. Microencapsulation or nanoencapsulation into particles may improve bioavailability profiles of cannabinoids and prevent degradation in gastric fluid as well as potential conversion from CBD to THC. Encapsulation of cannabinoids may result in particles of size 20-40 nm. Microencapsulation or nanoencapsulation may be by liposomal encapsulation, such that the cannabinoids are present inside particles having lipid walls. Other encapsulation methods may be used.

In embodiments, freeze dried cannabinoids may be in solid form obtained from freezing cannabis oil containing cannabinoids and subliming other components, leaving a solid having a high cannabinoid concentration. Solid cannabinoids may be effectively incorporated into a chewing composition by combining with other suitable solid carriers and embedding the resulting solid as a granule within the chewing gum composition.

Cannabinoids or derivatives thereof may provide pain relief, in particular neuropathic pain, and anti-inflammation effect. Post-herpetic neuralgia is characterized by intense pain of neuropathic nature in conjunction with inflammation, symptoms which may be alleviated by cannabinoids and/or derivatives thereof.

Cannabinoids or derivatives thereof may provide an increased concentration of dopamine in the brain. Restless leg syndrome relates to the dysfunction of the brain's basal ganglia circuits, which use dopamine. Cannabinoids or derivatives thereof may affect restless leg syndrome by modifying dopamine concentration in the brain.

In embodiments, the chewing gum composition may further comprise gabapentin. Gabapentin may be present in the chewing gum composition at 5-15% by weight based on the total weight composition. In a 2 g chewing gum piece, gabapentin may comprise 100-300 mg. Gabapentin is water soluble and may be incorporated into the chewing gum for sustained release.

In embodiments, gabapentin in the chewing gum composition may be provided in microencapsulated or nanoencapsulated form. Nanoencapsulation into particles of size 20-40 nm may improve bioavailability of gabapentin. Encapsulation may also aid with dissolution in the subject's oral cavity and transmucosal delivery mechanism. Methods to encapsulate gabapentin may be methods commonly used in the art, such as precision particle encapsulation, spray drying, or any other encapsulation technique like fluid bed coating.

Gabapentin reduces neuropathic pain by interaction with voltage-gated calcium channels, and thus reduces central neuropathic pain. Gabapentin's effect on neurotransmitters may also reduce symptoms of restless leg syndrome.

In embodiments, encapsulated gabapentin may be combined with encapsulated cannabinoids with at least one suitable carrier, such that encapsulated cannabinoids and gabapentin may be present in the chewing gum composition. The at least one carrier may act as a “binding matrix” to hold cannabinoids and gabapentin together in the chewing gum composition.

In embodiments, gum base provided for the chewing gun composition may be non-disintegrating. Gum base such as Gum powder PG 11 TA, Gum powder PG 11 TA New, Gum powder PG 5 TA, Gum powder PG 5 TA New, and Gum powder PG N12 TA may be used. Gum base may comprise 20-80% by weight of the composition.

At least one buffering agent may be included in this chewing gum composition. Suitable buffering agents may include acetates, glycinates, phosphates, carbonates, glycerophosphates, citrates, borates, and/or mixtures thereof. Buffering agents may be present at 5-35% by weight.

The chewing gum composition according to embodiments may have other ingredients to improve organoleptic properties. The chewing gum composition according to embodiments may include at least one flavoring agent and at least one sweetening agent.

In embodiments, ingredients such as certain flavoring agents may be included. Flavoring agents may include peppermint, spearmint, cinnamon, watermelon, licorice, cherry, orange, peach, and/or other suitable flavoring agents. Flavoring agents may be present in this chewing composition at 1-10% by weight.

Sweetening agents may include isomalt, sorbitol, maltitol, mannitol, stevia, xylitol, other suitable sweetening agents, and/or combinations thereof. Sweetening agents may be present at 1-65% of by weight of the composition according to embodiments. Certain food colorants may be included to improve the aesthetic appearance of the chewing gum composition.

The chewing gum composition according to embodiments may comprise ingredients for preservation such as citric acid. Additional ingredients to assist with powder flow and prevent the gum base from sticking to manufacturing surfaces may be included. Such ingredient may be silicon dioxide or magnesium stearate. Other ingredients for preservation and manufacturing management may also be used.

In embodiments, additional pharmaceutically acceptable excipients used in the chewing gum composition may be fillers, disintegrants, binders, or lubricants. Anti-oxidants such as ascorbyl palminate and sodium ascorbate may also be included. The chewing gum composition according to embodiments may comprise at least one pharmaceutically acceptable excipient and/or at least one anti-oxidant.

In embodiments, the chewing gum composition may further comprise a preservative to prevent degradation. Preservative used in the chewing gum composition according to embodiments may be citric acid.

In embodiments, the chewing gum composition may be made by a compressing process or by a hot process. In a compressing process, ingredients are mixed and compressed into the gum base using a compress machine. In a hot process, ingredients are mixed and heated before the gum base is poured in. The gum mixture is then molded and left to cure.

In embodiments, the chewing gum composition disclosed herein may be used for post-herpetic neuralgia or restless leg syndrome treatment. Cannabinoids and derivatives thereof and gabapentin in these chewing gums according to embodiments may be released in a controlled manner and absorbed by a subject via transmucosal delivery mechanism. A mammal, such as a human being, may chew the chewing gum composition according to embodiments 1-6 times a day to treat or alleviate symptoms of post-herpetic neuralgia or restless leg syndrome.

The chewing gum composition according to embodiments may be used in treatment of pain and/or chronic pain. A mammal, such as a human being, may chew the chewing gum composition according to embodiments as needed for treatment or alleviation of symptoms of pain.

### EXAMPLES

### Example 1

Chewing Gum Composition Preparation

Chewing gum compositions having 10 mg of THC and 300 mg of gabapentin are prepared by cold pressing. Ingredients listed below are obtained. Percentages are given in weight percentage.

TABLE 1

| Phase | Raw material | Percentage (%) |
|---|---|---|
| A1 | Isomalt | 16.50 |
| A2 | THC (nanoencapsulated) | 0.50 |
| A3 | Cellulose | 0.50 |
| A4 | Gabapentin (nanoencapsulated) | 15.00 |
| B1 | Gum base | 35.00 |
| B2 | Sorbitol | 10.00 |
| B3 | Maltitol | 5.00 |
| B4 | Citric acid | 0.50 |
| B5 | Magnesium stearate | 2.00 |
| B6 | Silicon dioxide | 0.40 |
| B7 | Xylitol | 5.54 |
| B8 | Stevia | 1.05 |
| B9 | Licorice | 4.00 |
| B10 | Spearmint | 4.00 |
| B11 | Colorants FD&C blue | 0.01 |
| | Total | 100.00 |

Step 1: Make a blend of A2, A3 and A4 into A1 to form Phase 1.

Step 2: Mix B1-B11 in a separate vessel until homogenous to form Phase 2.

Step 3: Use a double layer chewing gum machine to compress Phase 1 and Phase 2 together.

Cut the resulting chewing gum composition into pieces of 2 grams each.

Chewing gum compositions with a mass at about 2 grams and containing 10 mg of THC and 300 mg of gabapentin were prepared.

### Example 2

Chewing gum compositions having 40 mg of CBD and 200 mg of gabapentin are prepared. Ingredients listed below are obtained. Percentages are given in weight percentage.

TABLE 2

| Phase | Raw material | Percentage (%) |
|---|---|---|
| A1 | Gum base | 47.6 |
| A2 | Xylitol | 30 |
| A3 | Glycerine | 4.5 |
| B1 | Sacharrine | 0.4 |
| B2 | H20 | 1.5 |
| B3 | Gabapentin (encapsulated) | 10 |
| B4 | Citric acid | 0.5 |
| C1 | Peppermint aroma oil | 1.5 |
| A4 | Peppermint powder | 1.5 |
| C2 | CBD (encapsulated) | 2 |
| A5 | Cellulose | 0.5 |
| | Total | 100.00 |

Step 1: Heat the gum base (A1) to 90° C., then add A2-A5 to form Phase 1.

Step 2: Dissolve B1 and B4 in B2 to form Phase 2.

Step 3: Heat the peppermint oil (C1) to 60-70 ° C., then add C2 and B3 to form Phase 3.

Step 4: Add Phase 2 to Phase 1, stir vigorously and add Phase 3, stir for 7 minutes.

Step 5: Pour the gum mixture out and prepare chewing gum tablets by molding as need.

Chewing gums with a mass at about 2 grams and containing 40 mg of CBD and 200 mg of gabapentin were prepared.

Variations and modifications will occur to those of skill in the art after reviewing this disclosure. The disclosed features may be implemented, in any combination and sub-combination (including multiple dependent combinations and sub-combinations), with one or more other features described herein. The various features described or illustrated above, including any components thereof, may be combined or integrated in other systems. Moreover, certain features may be omitted or not implemented.

Examples of changes, substitutions, and alterations are ascertainable by one skilled in the art and could be made without departing from the scope of the information disclosed herein. All references cited are hereby incorporated by reference herein in their entireties and made part of this application.

What is claimed is:

1. A chewing gum composition comprising, based on total weight of the composition:
0.125 to 7.5% by weight of at least one cannabinoid;
5 to 15% by weight of gabapentin;
20 to 80% by weight of a gum base;
5 to 35% by weight of at least one buffering agent selected from the group consisting of acetates, glycinates, phosphates, carbonates, glycerophosphates, citrates, and borates;
1 to 10% by weight of at least one flavoring agent;
1 to 65% by weight of at least one sweetening agent selected from the group consisting of isomalt, sorbitol, stevia, maltitol, mannitol, and xylitol; and
at least one anti-oxidant selected from the group consisting of ascorbyl palminate and sodium ascorbate.

2. The chewing gum composition of claim 1, wherein the at least one cannabinoid is cannabidiol, cannabichromene, cannabigerol, cannabidivarin, derivatives thereof, or their acid metabolites.

3. The chewing gum composition of claim 1, wherein the at least one cannabinoid is Δ⁹-tetrahydrocannabinol.

4. The chewing gum composition of claim 3, wherein Δ⁹-tetrahydrocannabinol is present at 0.125 to 1% by weight of the total composition.

5. The chewing gum composition of claim 1, wherein the at least one cannabinoid is present in combination with at least one suitable carrier selected from the group consisting of sugar alcohol, microcrystalline cellulose derivatives, dextran, agarose, agar, pectin, alginate, xanthan, chitosan, and starch.

6. The chewing gum composition of claim 1, wherein the at least one cannabinoid is present in freeze dried form.

7. The chewing gum composition of claim 1, wherein the at least one cannabinoid is present in internal voids within a suitable solid carrier.

8. The chewing gum composition of claim 1, wherein the at least one cannabinoid is present in combination with a carrier in a granule within the gum matrix.

9. The chewing gum composition of claim 1, wherein the at least one cannabinoid is procured from natural sources or synthetic.

10. The chewing gum composition of claim 1, wherein the at least one flavoring agent is selected from the group consisting of peppermint, spearmint, cinnamon, licorice, cherry, orange, peach, and watermelon.

11. The chewing gum composition of claim 1, wherein the at least one cannabinoid and the gabapentin are provided as microencapsulated or nanoencapsulated particles.

12. The chewing gum composition of claim 11, wherein the nanoencapsulated or microencapsulated gabapentin or cannabinoid particles are liposomal particles.

13. The chewing gum composition of claim 1, further comprising at least one preservative and at least one antioxidant.

14. The chewing gum composition of claim 13, wherein the preservative is citric acid.

15. The chewing gum composition of claim 13, further comprising at least one pharmaceutically acceptable excipient selected from the group consisting of fillers, disintegrants, binders, and lubricants.

16. The chewing gum composition of claim 15, further comprising silicon dioxide or magnesium stearate.

17. A method to treat post-herpetic neuralgia in a mammal in need thereof, comprising administering to the mammal a chewing gum composition of claim 1.

18. The method of claim 17, wherein the mammal receives the chewing gum administration 1 to 6 times a day.

19. A method to treat restless leg syndrome in a mammal in need thereof, comprising administering to the mammal a chewing gum composition of claim 1.

20. The method of claim 19, wherein the mammal receives the chewing gum administration 1 to 6 times a day.
